(19) United States
(12) Patent Application Publication
(10) Pub. No.: US 2016/0270669 A1
(43) Pub. Date: Sep. 22, 2016

(54) DIAGNOSTIC IMAGING PROBE

(71) Applicant: TERUMO KABUSHIKI KAISHA, Tokyo (JP)

(72) Inventor: Masanori TOKIDA, Ashigarakami-gun (JP)

(73) Assignee: TERUMO KABUSHIKI KAISHA, Tokyo (JP)

(21) Appl. No.: 15/074,144

(22) Filed: Mar. 18, 2016

(30) Foreign Application Priority Data
Mar. 20, 2015 (JP) 2015-058260

(51) Int. Cl. A61B 5/02; A61B 8/12; A61B 8/13; A61M 25/00; A61B 5/00

(52) U.S. Cl. CPC A61B 5/02007; A61M 25/0082; A61B 5/0066; A61B 8/13; A61B 8/12; A61B 2562/12

(57) ABSTRACT

An imaging probe for diagnosis includes an imaging core that has an optical transceiver and an ultrasound transceiver, a first fixing unit that fixes the optical transceiver, a second fixing unit that fixes the ultrasound transceiver, and a connection member that connects the first fixing unit and the second fixing unit to each other. The connection member has bending rigidity which is lower than that of the first fixing unit and the second fixing unit.

### CROSS-REFERENCES TO RELATED APPLICATIONS

This application claims priority to Japanese Application No. 2015-058260 filed on Mar. 20, 2015, the entire content of which is incorporated herein by reference.

### TECHNICAL FIELD

The present disclosure relates to an imaging probe for diagnosis which is used to diagnose a body lumen such as a blood vessel and the like.

### BACKGROUND DISCUSSION

It is known to percutaneously treat a stenosis lesion or the like occurring inside a body lumen such as the blood vessel and the vascular channel. During such percutaneous treatment, in order to observe characteristics of the lesion or to observe a post-treatment state, a catheter for diagnosis which utilizes ultrasound, light, or the like is used to acquire a tomographic image of the inside of the body lumen.

In a case of intravascular ultrasound (IVUS) diagnosis, in general, a rotatable imaging core having an ultrasound transducer in a distal end of an insertion portion is disposed so as to perform rotary scanning (radial scanning) via a drive shaft or the like extending from the imaging core to an operator side drive unit.

In addition, in a case of optical coherence tomography (OCT) diagnosis utilizing a wavelength swept system, an imaging core is present in which an optical transceiver is attached to a distal end of an optical fiber, and is rotated via a drive shaft or the like extending from the imaging core to an operator side drive unit. While the imaging core is rotated, near-infrared light is emitted to a vascular lumen from the optical transceiver in the distal end, and the reflected light from body tissues is received so as to perform the radial scanning on the inside of the blood vessel. Then, based on interference light generated by causing interference between the received reflected light and reference light, a cross-sectional image of the blood vessel is generally extracted.

With OCT, a high-resolution image can be obtained, but the image only ranges from a vascular lumen surface to relatively shallow tissues. And while IVUS is inferior to OCT from a standpoint of obtainable image resolution, it is possible to obtain an image of vascular tissues which is deeper than those obtained with OCT. Therefore, in recent years, an imaging apparatus for diagnosis having an imaging core in which an IVUS function and an OCT function are combined with each other (imaging apparatus for diagnosis which comprises an ultrasound transceiver capable of transmitting and receiving ultrasound and an optical transceiver capable of transmitting and receiving light) has been proposed, for example, in JP-A-11-56752.

### SUMMARY

Here, in a case where an imaging apparatus for diagnosis has an imaging core in which an IVUS function and an OCT function are combined with each other, an ultrasound transceiver and an optical transceiver are mounted on a housing in a distal portion of the imaging core. However, when the ultrasound transceiver and the optical transceiver are disposed in the housing of the distal portion, a size of the distal portion increases, and a length thereof is also lengthened.

However, with the increased size of the housing of the distal portion or the lengthened length, the movement of the imaging core cannot follow a curved body lumen (for example, the blood vessel) and a curved catheter sheath. For example, the movement inside the catheter sheath will not be smooth, and in some cases, the distal portion will rub against an inner wall of the catheter sheath. This can cause the catheter not to function as desired. In addition, since the distal portion is rotated quickly during pullback, an excessive load is applied to the inner wall of the catheter sheath, thereby causing a possibility of damage to the catheter sheath. Furthermore, if the catheter sheath is damaged, there occurs a risk of damage to the blood vessel. In this regard, an object of the present disclosure is to improve the following ability of the imaging core in the curved body lumen and the curved catheter sheath.

In order to achieve the above-described object and other objects, the present disclosure describes an imaging probe for diagnosis which comprises an imaging core that has an optical transceiver and an ultrasound transceiver, comprising a first fixing unit that fixes the optical transceiver, a second fixing unit that fixes the ultrasound transceiver, and a connection member that connects the first fixing unit and the second fixing unit to each other, in which the connection member has bending rigidity which is lower than that of the first fixing unit and the second fixing unit. According to the present disclosure, it is possible to improve the following ability of an imaging core in a curved body lumen and a curved catheter sheath.

### BRIEF DESCRIPTION OF THE DRAWINGS

FIG. 1 illustrates an external configuration of an imaging apparatus for diagnosis according to an embodiment.

FIG. 2 illustrates an example of a structure of an imaging core and a structure of a catheter for accommodating the imaging core according to a first embodiment.

FIG. 3 illustrates a modification example of the structure of the imaging core according to the first embodiment.

FIG. 4 illustrates an example of a structure of an imaging core according to a second embodiment.

### DETAILED DESCRIPTION

Hereinafter, embodiments according to the present disclosure will be described in detail with reference to the accompanying drawings. Throughout the specification, the same reference numerals represent elements having the same configuration.

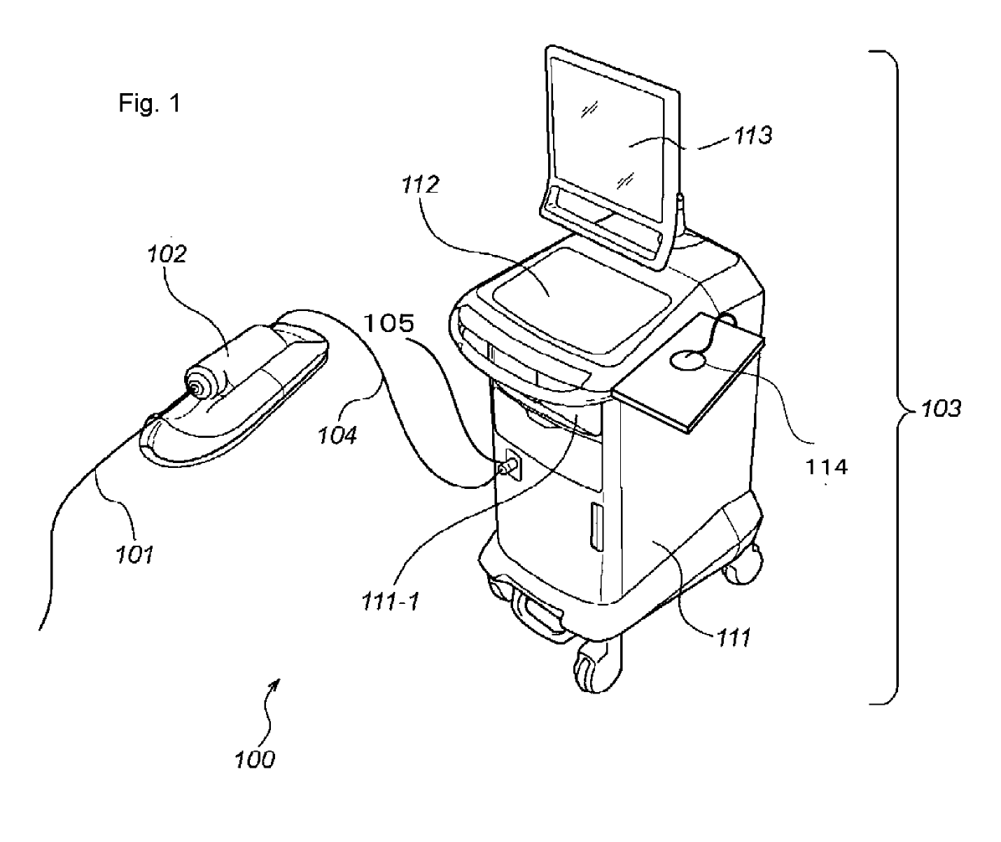

FIG. 1 illustrates an external configuration of an imaging apparatus for diagnosis 100 according to an embodiment. The imaging apparatus for diagnosis 100 according to the present embodiment has an IVUS function and an OCT function.

As illustrated in FIG. 1, the imaging apparatus for diagnosis 100 comprises an imaging probe for diagnosis 101, a scanner and pullback unit 102, and an operation control device 103. The scanner and pullback unit 102 and the operation control device 103 are connected to each other via a connector 105 by a cable 104 for accommodating a signal line or an optical fiber.

The imaging probe for diagnosis 101 is directly inserted into the blood vessel, and has an internally inserted catheter for accommodating an imaging core comprising an ultrasound transceiver which transmits ultrasound based on a pulse signal and receives a reflected wave from the inside of the blood vessel, and an optical transceiver which continuously transmits transmitted light (measurement light) into the blood vessel and continuously receives reflected light from the inside of the blood vessel. The imaging apparatus for diagnosis 100 measures a state inside the blood vessel by applying the imaging core.

The imaging probe for diagnosis 101 is detachably attached to the scanner and pullback unit 102. A motor incorporated in the scanner and pullback unit 102 is driven. In this manner, an operation in the axial direction and an operation in the rotation direction inside the blood vessel are regulated in the imaging core in the imaging probe for diagnosis 101 internally inserted into the catheter sheath. In addition, the scanner and pullback unit 102 acquires a signal of the reflected wave received by the ultrasound transceiver and the reflected light received by the optical transceiver inside the imaging core, and transmits both of these to the operation control device 103.

When carrying out measurement work, the operation control device 103 comprises a function for inputting various set values, and a function for displaying various blood vessel images after processing ultrasound data or optical interference data acquired by the measurement work.

In the operation control device 103, a main body control unit 111 generates line data, based on a signal of the reflected wave of the ultrasound obtained by the measurement work, and generates an ultrasound tomographic image through an interpolation process. Furthermore, the main body control unit 111 generates interference light data by causing interference between the reflected light from the imaging core and the reference light obtained by separating the light from a light source. The main body control unit 111 generates the line data, based on the interference light data, and generates a blood vessel tomographic image based on the optical interference through the interpolation process. The main body control unit 111 is embodied, for example, as a CPU executing a software program which is stored on a tangible, non-transitory computer-readable storage medium.

A printer and DVD recorder 111-1 prints a processing result in the main body control unit 111, or stores the processing result as data. A user inputs various set values and instructions via an operation panel 112. An LCD monitor 113 serves as a display device, and displays various tomographic images generated in the main body control unit 111. A mouse 114 serves as a pointing device (coordinate input device).

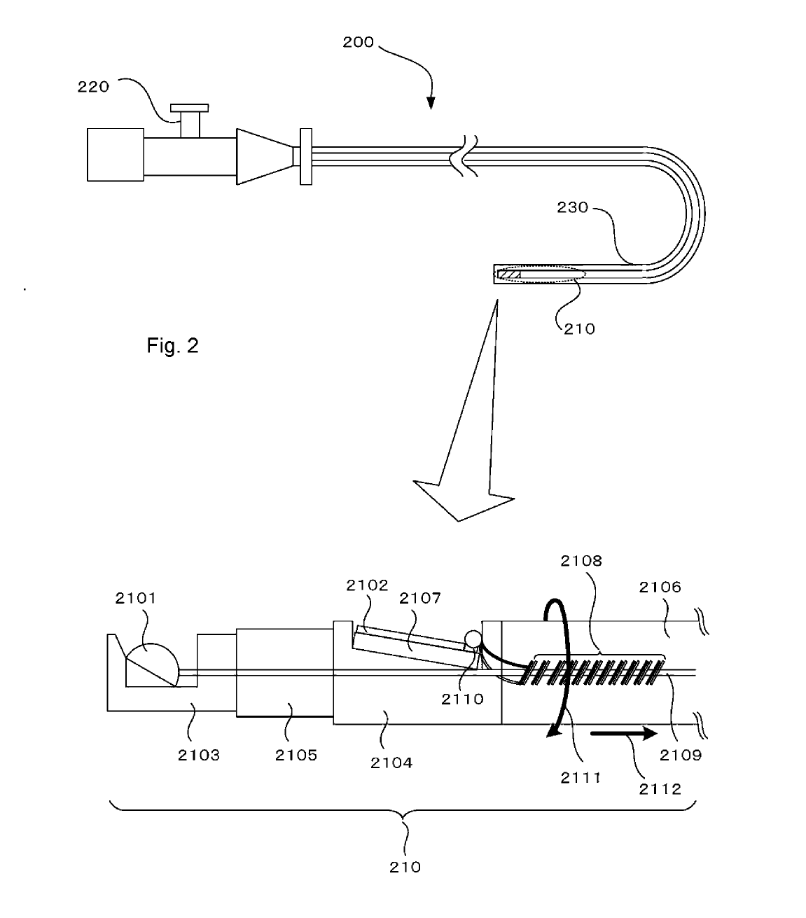

Next, referring to FIG. 2, a structure of an imaging core 210 and a structure of a catheter 200 for accommodating the imaging core 210 will be described. The reference numeral 200 in FIG. 2 represents a catheter according to the present embodiment. In addition, the catheter 200 corresponds to the imaging probe for diagnosis 101 in FIG. 1. An injection port 220 for injecting a transparent liquid (physiological saline solution or the like) into a catheter sheath 230 is disposed in the vicinity of a rear end (end portion connected to the pullback unit 102) in the catheter 200.

In addition, the catheter sheath 230 of the catheter 200 is configured to include a transparent material, and internally accommodates the imaging core 210 which is rotatable and movable along the catheter 200. The imaging core 210 comprises an optical transceiver 2101 and an ultrasound transceiver 2102. The ultrasound transceiver 2102 is supported by a backing member 2107. A first fixing unit 2103 for fixing the optical transceiver 2101 is disposed on a distal side of the imaging core 210, and a second fixing unit 2104 for fixing the ultrasound transceiver 2102 is disposed on a side in the pullback direction from the distal side. The second fixing unit 2104 is connected to a drive shaft 2106 by means of bonding, brazing (for example, soldering), or the like.

According to the present embodiment, a connection member 2105 and the drive shaft 2106 are configured to serve as an independent member. According to the present embodiment, the first fixing unit 2103 is a first housing, and the second fixing unit 2104 is a second housing which is different from the first housing. A connecting method of the first fixing unit 2103, the second fixing unit 2104, and the connection member 2105 can be changed depending on each material. As an example, when the second fixing unit 2104 serving as the second housing and the connection member 2105 are formed of a resin, the second fixing unit 2104 may be connected to the drive shaft 2106 by means of fusing. Alternatively, when the second fixing unit 2104 serving as the second housing and the connection member 2105 are formed of metal, the second fixing unit 2104 may be connected to the drive shaft 2106 by means of welding.

In addition, the connection member 2105 for connecting the first fixing unit 2103 and the second fixing unit 2104 to each other is disposed therebetween. The connection member 2105 is configured to include a material whose bending rigidity is lower than that of the first fixing unit 2103 and the second fixing unit 2104. The presence of the connection member 2105 allows for increased flexibility of the distal portion, thereby enabling the imaging core 210 to have improved following ability in a curved body lumen and the curved catheter sheath 230. As the connection member 2105, for example, it is possible to use an elastic member such as rubber and the like or a member having the same material as the drive shaft 2106.

The first fixing unit 2103 and the second fixing unit 2104 are manufactured by mainly using stainless steel (for example, SUS304, SUS303, SUS316, and SUS316L) or the like, and a Young's modulus E thereof is approximately 200 (GPa). In addition, the first fixing unit 2103 and the second fixing unit 2104 have a pipe shape in which an inner diameter is approximately 0.5 (mm) and an outer diameter is 0.6 (mm). Therefore, a secondary moment I in a cross section of each unit can be calculated as 3.3×10 (mm⁴). Accordingly, bending rigidity El of each unit is 6.6×10−4 (N.m²). The catheter sheath 230 is used in the curved blood vessel having the curvature radius of approximately 15 (mm). However, if the catheter sheath 230 is a member having high bending rigidity such as a stainless steel pipe and the imaging core 210 is longer than the conventional one, the imaging core 210 is not bent inside the catheter sheath 230. Consequently, the following ability in the curve is degraded. Therefore, the connection member 2105 is configured to include a material having a smaller value than the value of the bending rigidity. In this manner, when the imaging core portion enters the curved portion of the blood vessel, the imaging core is selectively curved in the connection member portion having lower bending rigidity. Accordingly, it is possible to improve the following ability in the catheter sheath 230.

As a specific example of the connection member 2105, if a rubber-based material (Young's modulus is approximately 0.01 (GPa) to 0.1 (GPa)), a nylon-based material (Young's modulus is approximately 3 (GPa) to 7 (GPa)), or the like which has a smaller Young's modulus than each fixing unit is used, the connection member 2105 can have a two-to-four digit decrease in the value of the bending rigidity. Of course, improvements to the drive shaft to improve its following ability in the curve could also be adapted for use in the connection member 2105.

In addition, the first fixing unit 2103 and the connection member 2105, and the connection member 2105 and the second fixing unit 2104 are similarly connected to each other by means of bonding, brazing (for example, soldering), fusing, welding, or the like.

The drive shaft 2106 is formed of a flexible material having characteristics which can transmit rotation power well, and for example, the drive shaft 2106 is configured to include a multiple-multilayer-tightly wound coil or the like formed of a metal wire such as a stainless steel wire and the like. In this case, a signal line 2108 and an optical fiber 2109 can be accommodated inside the drive shaft 2106. An end portion of the signal line 2108 is bonded to an electrode 2110 of the ultrasound transceiver 2102 on the backing member 2107 by means of soldering. Such provision of the backing member 2107 can help prevent reflection from a rear surface side of the ultrasound transceiver 2102. Accordingly, it is possible to prevent reflection from portions other than the vascular lumen surface.

The first fixing unit 2103 and the second fixing unit 2104 have a partially cutout portion. The ultrasound transceiver 2102 and the optical transceiver 2101 transmit and receive ultrasound or light via the cutout portion.

The ultrasound transceiver 2102 emits the ultrasound in accordance with a pulse signal applied from the signal line 2108, detects a reflected wave from vascular tissues, and transmits the reflected wave to the signal line 2108, as an electrical signal.

The optical transceiver 2101 is disposed in an end portion of the optical fiber 2109, and forms a hemispherical shape obtained by cutting a spherical body along a vertical plane in FIG. 2 at an angle of approximately 45 degrees. A mirror portion is formed on an inclined surface thereof. In addition, since the optical transceiver 2101 has the hemispherical shape, the optical transceiver 2101 also comprises a lens function. Light supplied via the optical fiber 2109 is reflected on the mirror portion, and is emitted toward the vascular tissues. Then, the reflected light is received from the vascular tissues, is reflected on the mirror portion, and the reflected light is caused to return to the optical fiber 2109.

When scanning is performed, in response to the driving of a radial scanning motor of the pullback unit 102, the drive shaft 2106 rotates along an arrow 2111, and moves along an arrow 2112. As a result, while the ultrasound transceiver 2102 fixed to the second fixing unit 2104 and the optical transceiver 2101 fixed to the first fixing unit 2103 rotate and move in the axial direction, the ultrasound is emitted, the reflected wave is detected, the light is emitted, and the reflected light is detected.

In addition, the present embodiment adopts a configuration in which the outer diameter of the first fixing unit 2103 disposed on the distal side of the imaging core 210 is smaller than the outer diameter of the second fixing unit 2104 as illustrated in FIG. 2. In addition, a configuration may also be adopted which satisfies an expression of the outer diameter of the first fixing unit 2103≦the outer diameter of the connection member 2105≦the outer diameter of the second fixing unit 2104. When the distal side of the imaging core 210 is configured to have the further decreased outer diameter, it is possible to reduce the possibility that the inner wall of the catheter sheath 230 and the distal end of the imaging core 210 may come into contact with each other. Therefore, it is possible to further improve the following ability of the imaging core 210 in the curved body lumen and the curved catheter sheath 230.

Conventionally, the optical transceiver 2101 and the ultrasound transceiver 2102 have been arranged in one housing. However, according to the present embodiment, the optical transceiver 2101 and the ultrasound transceiver 2102 are respectively arranged in a separate fixing unit (housing). Accordingly, the size of each housing can be decreased compared to the conventional housing. Therefore, it is possible to reduce the possibility that the inner wall of the catheter sheath 230 and the distal end of the imaging core 210 may come into contact with each other. Thus, it is possible to further improve the following ability of the imaging core 210 in the curved body lumen and the curved catheter sheath 230.

As described above, the imaging probe for diagnosis 101 according to the present embodiment comprises the imaging core 210 having the optical transceiver 2101 and the ultrasound transceiver 2102. The imaging probe for diagnosis 101 comprises the first fixing unit 2103 for fixing the optical transceiver 2101, the second fixing unit 2104 for fixing the ultrasound transceiver 2102, and the connection member 2105 for connecting the first fixing unit 2103 and the second fixing unit 2104 to each other. The connection member 2105 has lower bending rigidity than the first fixing unit 2103 and the second fixing unit 2104.

Since the connection member having the lower bending rigidity is disposed in this way, it is possible to improve the following ability of the imaging core in the curved body lumen and the curved catheter sheath. In addition, since the divided housing (fixing unit) is arranged, the size of the housing (fixing unit) in the distal end can be decreased, and the length can be shortened. Therefore, it is possible to further improve the following ability.

In the present embodiment, an example has been described in which the first fixing unit 2103 (optical transceiver 2101) is arranged on the distal side of the imaging core 210 and the second fixing unit 2104 (ultrasound transceiver 2102) is arranged on the side in the pullback direction. In general, the reason is that the size of the lens included in the optical transceiver 2101 is smaller than the size of the ultrasound transducer included in the ultrasound transceiver 2102. If the first fixing unit 2103 (optical transceiver 2101) is arranged on the distal side, the distal portion can be further miniaturized, and the operation can be more smoothly performed. Moreover, it is possible to improve the following ability of the imaging core. However, the present disclosure is also applicable to a configuration in which the second fixing unit 2104 (ultrasound transceiver 2102) is arranged on the distal side and the first fixing unit 2103 (optical transceiver 2101) is arranged on the side in the pullback direction.

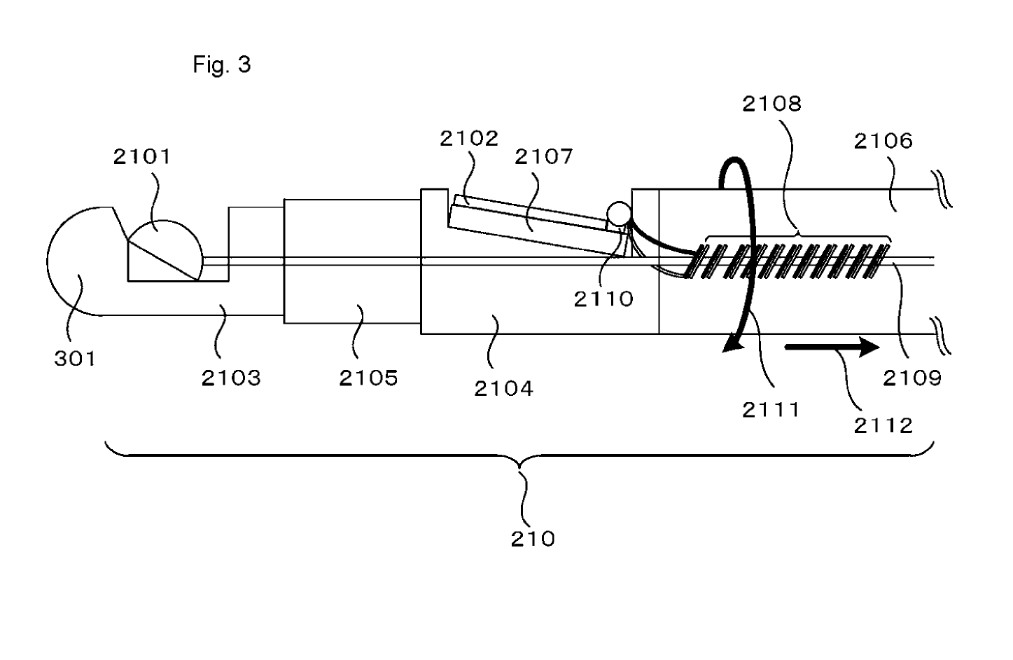

Subsequently, referring to FIG. 3, a modification example of the imaging probe for diagnosis 101 according to the present embodiment will be described. As illustrated in FIG. 3, the distal portion of the first fixing unit 2103 has a curved shape. For example, a configuration having a hemispherical shape may be adopted. Alternatively, without being limited to the hemispherical shape, the first fixing unit 2103 disposed on the distal side of the imaging core 210 may be configured so that the outer diameter on the distal side is equal to or smaller than the outer diameter on the connection member 2105 side. The first fixing unit 2103 may be configured so that the distal end has the further decreased outer diameter. For example, the first fixing unit 2103 may be configured to include multiple cylinders whose outer diameters are decreased at multiple stages.

The first fixing unit 2103 is configured so that the distal end has the further decreased outer diameter. In this manner, it is possible to further reduce the possibility that the inner wall of the catheter sheath 230 and the distal end of the imaging core 210 may come into contact with each other. Thus, it is possible to further improve the following ability of the imaging core 210 in the curved body lumen and the curved catheter sheath 230.

In the first embodiment, an example has been described in which the first fixing unit 2103 and the second fixing unit 2104 are configured to respectively serve as a separate housing, and in which both of these are connected to each other by the connection member 2105 whose bending rigidity is lower than both of these.

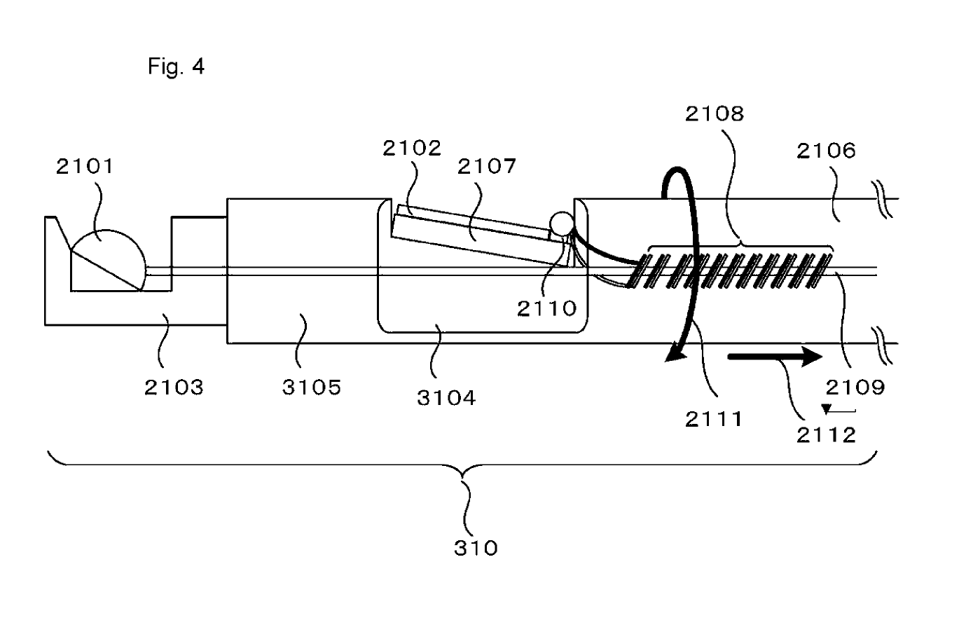

In contrast, in a second embodiment, an example will be described in which a connection member is configured to serve as a part of the drive shaft, and in which the second fixing unit is configured to be brazed to a window portion disposed in the drive shaft. FIG. 4 illustrates an example of a structure of an imaging core according to the second embodiment of the present disclosure. Description will be omitted with regard to the same configuration elements as the configuration elements described in the first embodiment.

In an imaging core 310 according to the present embodiment, a connection member 3105 corresponding to the connection member 2105 according to the first embodiment is configured to serve as a part of the drive shaft 2106. That is, similarly to the drive shaft 2106, the connection member 3105 is flexible, since the connection member 3105 is configured to include a multiple-multilayer-tightly wound coil or the like formed of a metal wire.

Then, a second fixing unit 3104 is brazed to a window portion disposed in the drive shaft 2106, and is configured to serve as one rigid section. In this manner, the second fixing unit 3104 has the same function as that of the housing. The first fixing unit 2103 is the housing disposed on the distal side of the imaging core 310, and has the same configuration as that according to the first embodiment.

According to this configuration, the connection member 3105 can be configured to serve as an elastic member having the same material as that of the drive shaft 2106. In this manner, it is possible to improve the following ability of the imaging core in the curved body lumen and the curved catheter sheath. Furthermore, since only one housing is disposed in the distal end, the configuration contributes to reduced housing materials.

Hitherto, the embodiments according to the present disclosure have been described. However, a part of the configuration described in the first embodiment and the configuration described in the second embodiment may be combined with each other. For example, as described in the first embodiment, the second embodiment may also adopt a configuration which satisfies an expression of the outer diameter of the first fixing unit 2103≦the outer diameter of the connection member 3105≦the outer diameter of the second fixing unit 3104. In addition, the first fixing unit 2103 may be configured so that the distal end has the further decreased outer diameter.

The detailed description above describes a diagnostic imaging probe. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An imaging probe for diagnosis which comprises an imaging core that has an optical transceiver and an ultrasound transceiver, comprising:
a first fixing unit that fixes the optical transceiver;
a second fixing unit that fixes the ultrasound transceiver; and
a connection member that connects the first fixing unit and the second fixing unit to each other,
wherein the connection member has bending rigidity which is lower than that of the first fixing unit and the second fixing unit.

2. The imaging probe for diagnosis according to claim 1, further comprising:
a drive shaft that is connected to the second fixing unit.

3. The imaging probe for diagnosis according to claim 2,
wherein the connection member and the drive shaft are configured to serve as an independent member.

4. The imaging probe for diagnosis according to claim 1,
wherein the first fixing unit is a first housing, and the second fixing unit is a second housing which is different from the first housing.

5. The imaging probe for diagnosis according to claim 2,
wherein the first fixing unit is a first housing, and the second fixing unit is a second housing which is different from the first housing.

6. The imaging probe for diagnosis according to claim 3,
wherein the first fixing unit is a first housing, and the second fixing unit is a second housing which is different from the first housing.

7. The imaging probe for diagnosis according to claim 1,
wherein the first fixing unit and the connection member, and the connection member and the second fixing unit are connected to each other by means of any one of bonding, brazing, fusing, and welding.

8. The imaging probe for diagnosis according to claim 2,
wherein the first fixing unit and the connection member, and the connection member and the second fixing unit are connected to each other by means of any one of bonding, brazing, fusing, and welding.

9. The imaging probe for diagnosis according to claim 3,
wherein the first fixing unit and the connection member, and the connection member and the second fixing unit are connected to each other by means of any one of bonding, brazing, fusing, and welding.

10. The imaging probe for diagnosis according to claim 4,
wherein the first fixing unit and the connection member, and the connection member and the second fixing unit are connected to each other by means of any one of bonding, brazing, fusing, and welding.

11. The imaging probe for diagnosis according to claim 5,
wherein the first fixing unit and the connection member, and the connection member and the second fixing unit are connected to each other by means of any one of bonding, brazing, fusing, and welding.

12. The imaging probe for diagnosis according to claim 6,
wherein the first fixing unit and the connection member, and the connection member and the second fixing unit are connected to each other by means of any one of bonding, brazing, fusing, and welding.

13. The imaging probe for diagnosis according to claim 2,
wherein the connection member is configured to serve as a part of the drive shaft, and
wherein the second fixing unit is configured to be brazed to a window section disposed in the drive shaft.

14. The imaging probe for diagnosis according to claim 13,
wherein the first fixing unit is a housing disposed on a distal side of the imaging core.

15. The imaging probe for diagnosis according to claim 1,
wherein an outer diameter of the first fixing unit disposed on the distal side of the imaging core is smaller than an outer diameter of the second fixing unit.

16. The imaging probe for diagnosis according to claim 1,
wherein in the first fixing unit disposed on the distal side of the imaging core, an outer diameter on the distal side is equal to or smaller than an outer diameter on the connection member side.

17. The imaging probe for diagnosis according to claim 16,
wherein an end portion of the first fixing unit has a hemispherical shape.

18. The imaging probe for diagnosis according to claim 1,
wherein the connection member is an elastic member.
